(19) United States
(12) Patent Application Publication
(10) Pub. No.: US 2026/0325116 A1
(43) Pub. Date: Oct. 22, 2026

(54) SPERM COLLECTION DEVICE

(71) Applicant: Changhu ZHAN, Shenzhen City (CN)

(72) Inventor: Changhu ZHAN, Shenzhen City (CN)

(21) Appl. No.: 19/326,700

(22) Filed: Sep. 11, 2025

(30) Foreign Application Priority Data
Aug. 22, 2025 (CN) 202521804601.8

(51) Int. Cl. A61B 10/00

(52) U.S. Cl. CPC A61B 10/0058; A61H 19/32

(57) ABSTRACT

The present disclosure discloses a sperm collection device, comprising a housing, a sleeve, a drive assembly, two connection housings, two handles, and a control device. The housing forms a movable space; the sleeve is arranged within the movable space and forms a receiving space for accommodating the male genitalia; the drive assembly is arranged within the housing and drives the sleeve to perform linear reciprocating motion within the movable space; the two connection housings are respectively connected to both sides of the outer surface of the housing; the handles are rotatably connected to the connection housings around their axes, and the angle between the axis of the handles and the longitudinal axis of the housing is acute; the control device is arranged within the connection housings and/or the housing, electrically connected to the drive assembly.

### CROSS-REFERENCE TO RELATED APPLICATIONS

The present disclosure claims priority of the Chinese invention patent application with an application number of CN2025218046018, an invention title of ‘a sperm collection device’ and filed on Aug. 22, 2025.

### TECHNICAL FIELD

The present disclosure relates to the technical field of medical devices, and more particularly, to a sperm collection device.

### BACKGROUND OF THE DISCLOSURE

In clinical medicine, semen analysis is the most basic and primary examination method for judging and assessing male fertility. To perform semen analysis, male semen must first be collected, and therefore, the medical electronic device—the sperm collection device—has become a standard electronic device in major relevant hospital departments.

Typically, a sperm collection device includes a housing, a soft sleeve installed inside the housing and forming a cavity within the housing, and a motor used to drive the soft sleeve to move, thereby massaging and stimulating the penis inserted into the cavity of the soft sleeve. The housing of such products is generally large in size, making it difficult for users to hold, and it is very inconvenient to use. Moreover, when users need to adjust the movement amplitude and frequency of the soft sleeve, they must frequently press buttons, which makes it inconvenient to use.

Therefore, the present disclosure provides a sperm collection device that effectively solves the above problems, featuring a simple structure. The user can hold the handle to use the device, and can adjust the movement amplitude and frequency of the soft sleeve by rotating the handle, thereby providing a better user experience.

### SUMMARY OF THE DISCLOSURE

In order to overcome the deficiencies of the prior art, the present disclosure provides a sperm collection device with a simple structure and good sealing effect.

The technical solution adopted by the present disclosure to solve its technical problems is as follows:

A sperm collection device, including:
a housing, the housing forming a movable space;
a sleeve, the sleeve being disposed within the movable space and forming a receiving space for accommodating the male genitalia;
a drive assembly, the drive assembly being disposed within the housing and driving the sleeve to perform linear reciprocating motion within the movable space;
two connection housings, the two connection housings being respectively connected to both sides of the outer surface of the housing;
two handles, the handles being rotatably connected to the connection housings around their axes, and the angle between the axis of the handles and the longitudinal axis of the housing being an acute angle;
a control device, the control device being arranged within the connection housings and/or the housing, the control device being electrically connected to the drive assembly, the control device detecting the rotation angle of at least one of the handles and controlling the rotation direction and speed of the drive assembly based on the rotation angle, to control the stroke and/or frequency of the reciprocating motion of the sleeve.

As an improvement of the present disclosure, the angle between the axis of the handle and the vertical axis of the housing is between 40° and 80°.

As an improvement of the present disclosure, the control device detects the rotation angle of one of the handles and controls the rotation direction and speed of the drive assembly based on the rotation angle to control the stroke of the reciprocating motion of the sleeve, the control device detecting the rotation angle of the other handle and controlling the rotation direction and speed of the drive assembly based on the rotation angle to control the frequency of the reciprocating motion of the sleeve.

As an improvement of the present disclosure, the handle is provided with a magnet, the connection housing is provided with a Hall sensor, the Hall sensor being oriented towards the magnet, the Hall sensor being used to detect the magnetic field of the magnet and output a voltage signal to the control device.

As an improvement of the present disclosure, the handle is provided with an axial hole, the connection housing is provided with a rotating shaft, the rotating shaft being inserted into the axial hole and rotatable within the axial hole.

As an improvement of the present disclosure, the rotating shaft is provided with an annular protrusion, and the inner wall of the axial hole is provided with an annular groove, the annular protrusion being inserted into the annular groove.

As an improvement of the present disclosure, the free end of the rotating shaft is provided with a deformation slot.

As an improvement of the present disclosure, the connection housing is provided with a limiting block, and the handle is provided with an arcuate limiting slot, the limiting block being inserted into the limiting slot and being slidable within the limiting slot.

As an improvement of the present disclosure, the control device includes a main circuit board and a button circuit board, the main circuit board being arranged within the housing, and the button circuit board being arranged within the connection housing, the main circuit board being electrically connected to the button circuit board.

As an improvement of the present disclosure, it further comprises a trigger button, the trigger button being connected to the connection housing and rotating between a trigger position and a release position, the pressing portion of the trigger button extending out of the connection housing, and when the pressing portion of the trigger button is pressed by an external force, the trigger button rotates to the trigger position, and the trigger button presses the key contacts on the button circuit board.

As an improvement of the present disclosure, the connection housing is provided with an elastic member, one end of the elastic member abutting against the connection housing, and the other end of the elastic member abutting against the trigger button, the elastic member causing the trigger button to have a tendency to move towards the release position.

As an improvement of the present disclosure, the drive assembly includes a motor, a conveyor track, a driven gear, and a connecting block, the motor and the driven gear being disposed within the housing, the conveyor track meshing with the output end of the motor and the driven gear, one end of the connecting block being fixedly connected to the conveyor track, and the other end of the connecting block extending out of the housing and connecting to the sleeve.

As an improvement of the present disclosure, the housing further includes two sliding rods, the two sliding rods being respectively positioned on both sides of the connecting block, the connecting block being slidably connected to the sliding rods.

As an improvement of the present disclosure, the drive assembly further includes a driving gear, the motor being fixedly connected to one side of the inner wall of the housing, the output shaft of the motor being rotatably connected to the other side of the inner wall of the housing, the driving gear being connected to the middle of the output shaft of the motor, and the conveyor track meshing with the driving gear.

As an improvement of the present disclosure, the housing further includes an output shaft fixing member, the output shaft fixing member being fixedly connected to the inner wall of the housing, the output shaft fixing member being connected to the output shaft of the motor to prevent radial movement of the output shaft of the motor.

As an improvement of the present disclosure, the outer surface of the housing is provided with a connecting groove, the connection housing being detachably inserted into the connecting groove.

As an improvement of the present disclosure, the connection housing is provided with a first electrical connection terminal facing the housing, and the connecting groove is provided with a second electrical connection terminal, the first electrical connection terminal being electrically connected to the second electrical connection terminal.

As an improvement of the present disclosure, the connection housing is connected to the housing and rotates between a use position and a storage position.

As an improvement of the present disclosure, the connection housing is provided with a third electrical connection terminal facing the housing, and the connecting groove is provided with a fourth electrical connection terminal, the third electrical connection terminal being electrically connected to the fourth electrical connection terminal.

The beneficial effects of the present disclosure are:

When in use, the drive assembly works to drive the sleeve to perform linear reciprocating motion within the movable space, thereby massaging and stimulating the penis inserted into the receiving space; in addition, the user can rotate the handle, and the control device detects the rotation angle of the handle and outputs a corresponding electrical signal based on the rotation angle of the handle, which changes the rotation speed or the direction of rotation of the drive assembly, thereby controlling the stroke and/or frequency of the reciprocating motion of the sleeve. For example, rotating the left handle can adjust the rotation speed of the drive assembly, thereby increasing the frequency of the reciprocating motion of the sleeve. Rotating the right handle can adjust the speed of the rotation direction of the drive assembly, thereby changing the stroke of the reciprocating motion of the sleeve. The user can freely adjust the speed and amplitude of the sleeve's motion according to their needs, thus providing a better user experience.

### BRIEF DESCRIPTION OF DRAWINGS

In order to more clearly illustrate the technical solutions in the embodiments of the present disclosure, the following provides a brief introduction to the drawings that will be used in the description of the embodiments. The drawings described below are only some embodiments of the present disclosure. For those skilled in the art, other drawings can also be derived from these based on the drawings without any creative effort. In addition, the drawings are not to scale, and the relative sizes of various components are drawn illustratively, not necessarily according to actual proportions.

The present disclosure will be further described below in conjunction with the drawings and embodiments.

FIG. 1 is a schematic overall structural diagram from one angle of the first embodiment of the present disclosure;

FIG. 2 is a schematic overall structural diagram from another angle of the first embodiment of the present disclosure;

FIG. 3 is the first cross-sectional view of the first embodiment of the present disclosure;

FIG. 4 is the second cross-sectional view of the first embodiment of the present disclosure;

FIG. 5 is an enlarged view of the circle A in FIG. 4;

FIG. 6 is a cross-sectional view of the handle portion in the first embodiment of the present disclosure;

FIG. 7 is a schematic overall structural diagram of the second embodiment of the present disclosure;

FIG. 8 is a schematic exploded structural diagram from one angle of the second embodiment of the present disclosure;

FIG. 9 is a schematic exploded structural diagram from another angle of the second embodiment of the present disclosure;

FIG. 10 is an enlarged view of the circle B in FIG. 8;

FIG. 11 is an enlarged view of the circle C in FIG. 9;

FIG. 12 is a schematic overall structural diagram of the first state of the third embodiment of the present disclosure;

FIG. 13 is a schematic overall structural diagram of the second state of the third embodiment of the present disclosure.

### DETAILED DESCRIPTION OF THE EMBODIMENT

In order to make the above objectives, features, and advantages of this application more apparent and easier to understand, the following provides a detailed description of the specific embodiments of this application in combination with the accompanying drawings. In the description below, many specific details are provided to facilitate a full understanding of this application. However, this application can be implemented in many other ways different from those described herein, and those skilled in the art may make similar improvements without departing from the spirit of this application. Therefore, this application is not limited by the specific embodiments disclosed below.

In the description of this application, it should be understood that, if the terms “center,” “longitudinal,” “lateral,” “length,” “width,” “thickness,” “top,”“bottom,” “front,” “rear,” “left,” “right,” “vertical,” “horizontal,” “top,” “bottom,” “inside,” “outside,” “clockwise,” “counterclockwise,” “axial,” “radial,” “circumferential,” etc., are used, these terms indicate the orientation or positional relationship based on the orientation or positional relationship shown in the accompanying drawings. These terms are only for the convenience of describing this application and simplifying the description, and do not indicate or imply that the device or component referred to must have a specific orientation, structure, or operation. Therefore, they should not be understood as limiting this application.

Additionally, when the terms “first,” “second” appear, these terms are used only for description purposes and should not be understood as indicating or implying relative importance or implying the number of technical features referred to. Accordingly, features with “first” and “second” may explicitly or implicitly include at least one of the respective feature. In the description of this application, if the term “multiple” is used, the meaning of “multiple” is at least two, such as two, three, etc., unless explicitly limited otherwise.

In this application, unless otherwise clearly stated or defined, if terms such as “installed,” “connected,” “fixed,” etc., are used, these terms should be understood broadly. For example, it could be a fixed connection or a detachable connection, or integrated; it could be a mechanical connection or an electrical connection; it could be directly connected or indirectly connected through an intermediary medium. It could refer to a connection or interaction relationship between two components, unless clearly limited otherwise. For ordinary technicians in this field, they can understand the specific meaning of these terms in this application based on the specific situation.

In this application, unless otherwise clearly stated or defined, when a first feature is described as being “on,” “under,” etc., in relation to a second feature, it can mean that the first and second features directly contact or are in indirect contact through an intermediary medium. Also, when the first feature is described as being “on top of,” “above,” or “on” the second feature, it may mean that the first feature is directly above or obliquely above the second feature, or it simply means that the first feature is at a higher horizontal level than the second feature. When the first feature is described as being “under,” “below,” or “beneath” the second feature, it may mean that the first feature is directly beneath or obliquely beneath the second feature, or it simply means that the first feature is at a lower horizontal level than the second feature.

It should be noted that if a component is referred to as being “fixed to” or “set on” another component, it can either be directly on the other component or could also exist through a centered component. If a component is said to be “connected” to another component, it may be directly connected to the other component or possibly also connected via a centered component. As used in this application, the terms “vertical,” “horizontal,” “top,” “bottom,” “left,” “right,” and similar expressions are only for descriptive purposes and do not indicate the only possible implementation.

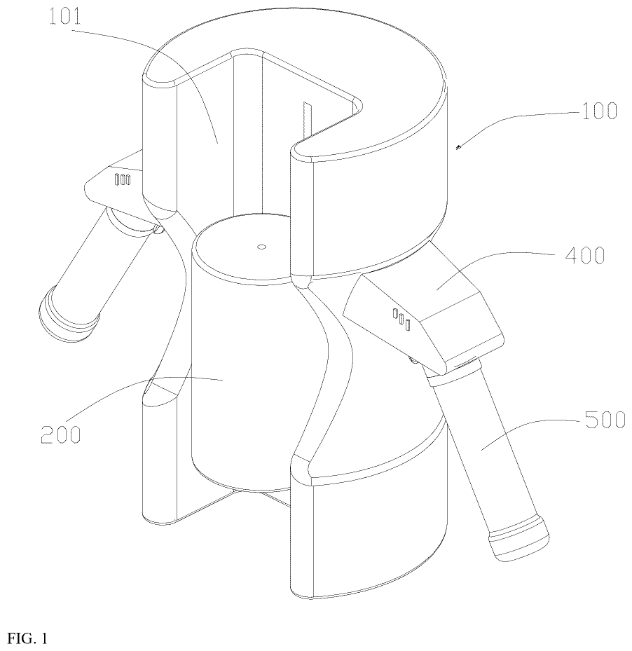

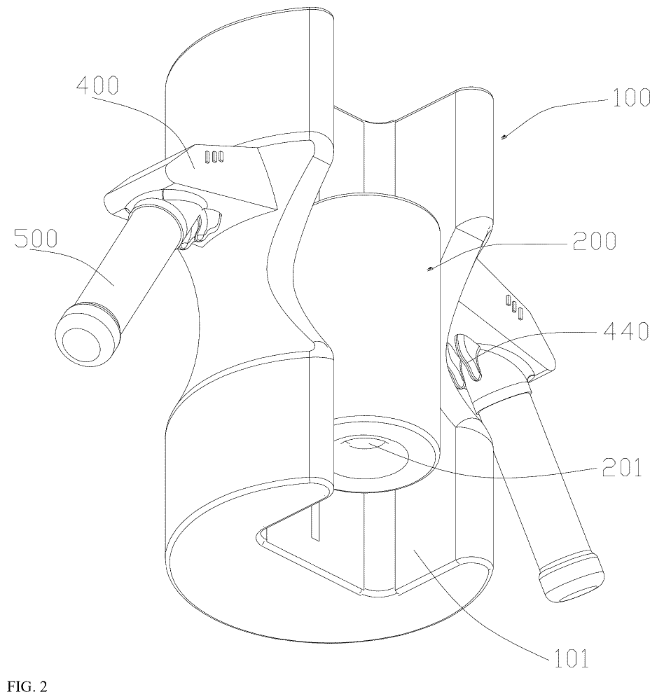

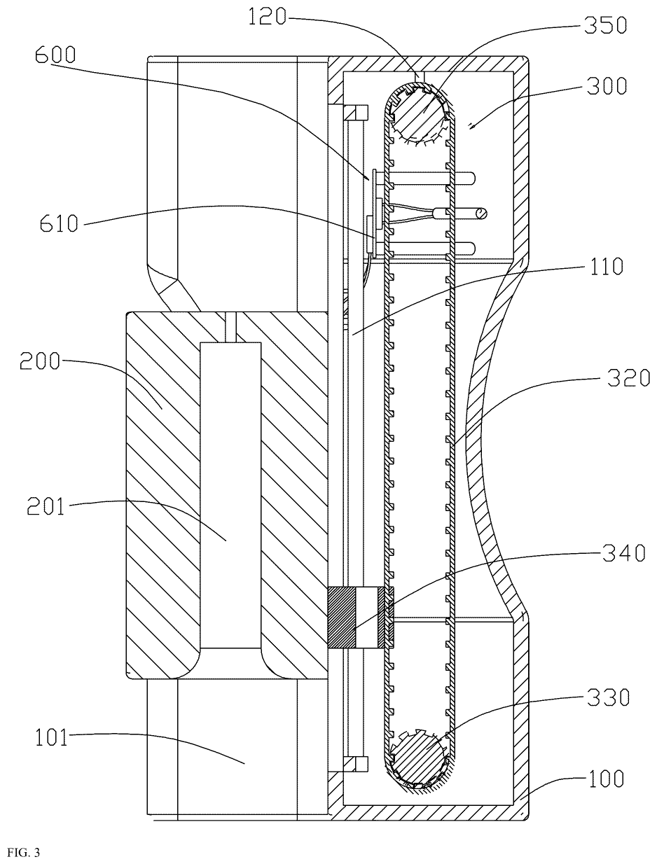

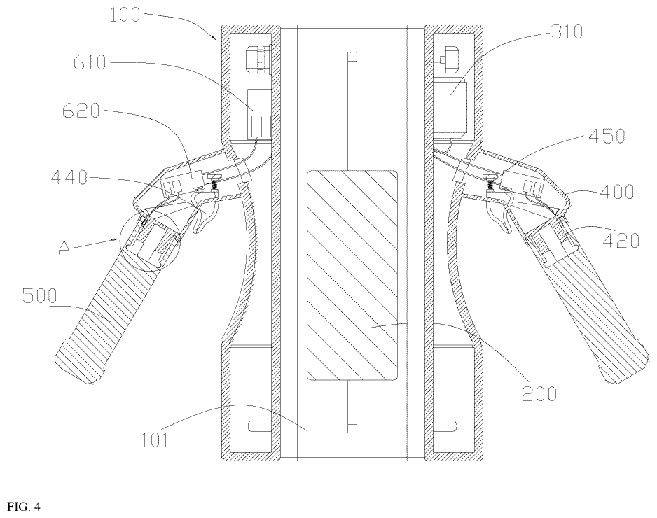

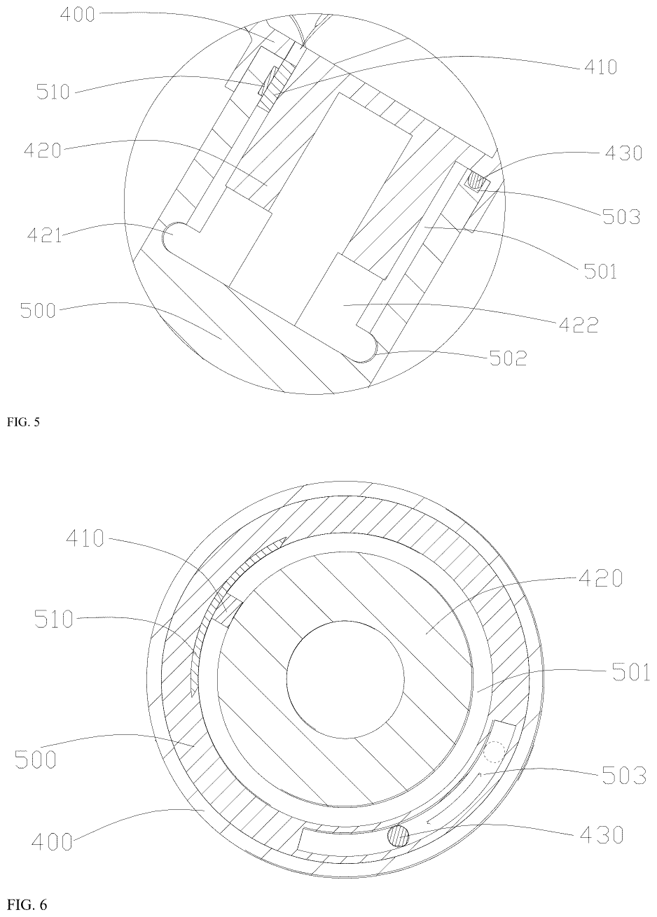

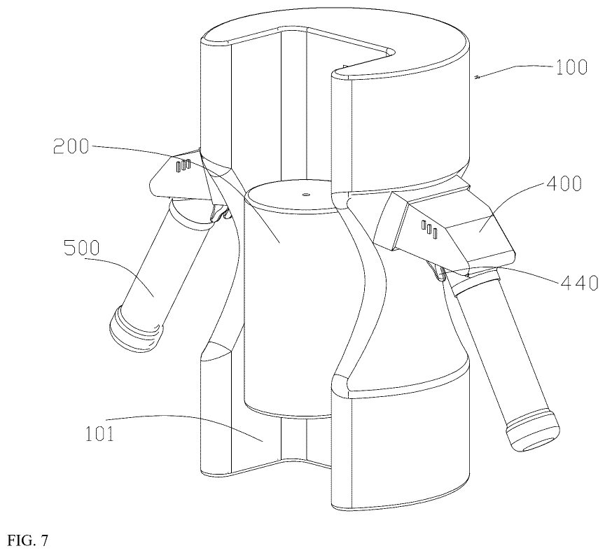

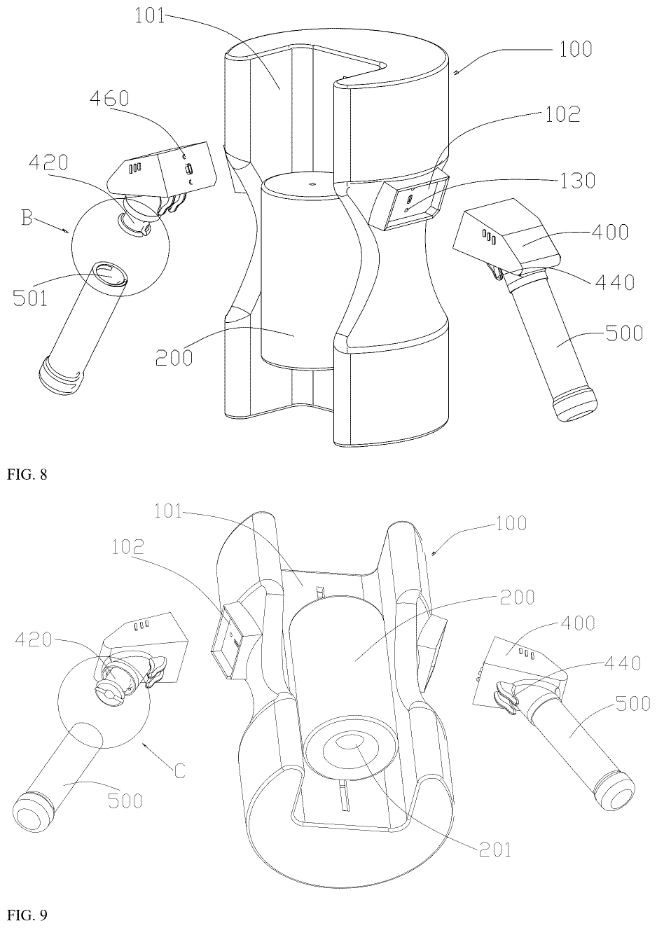

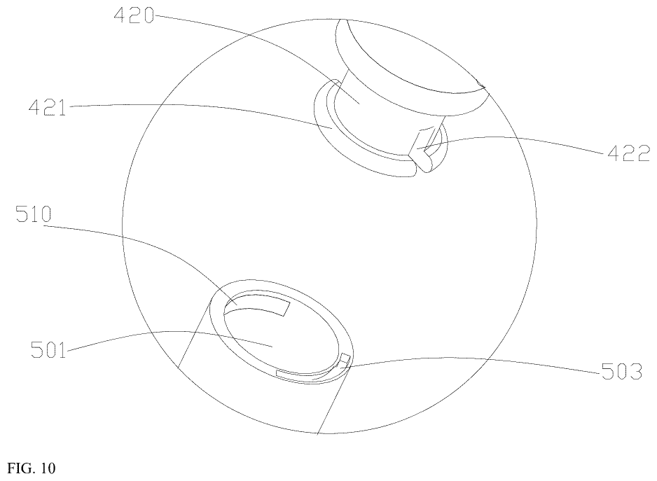

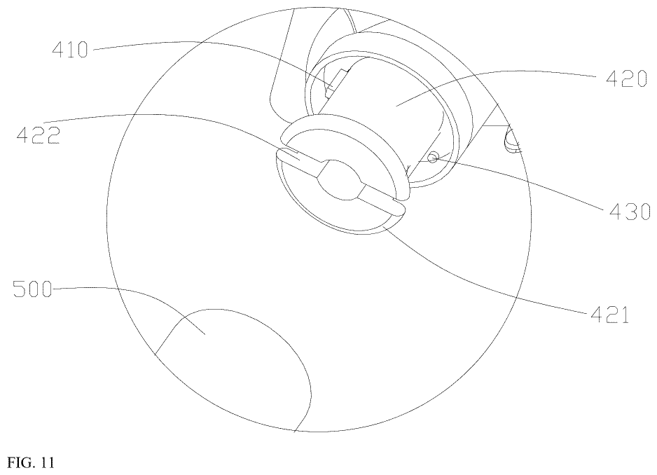

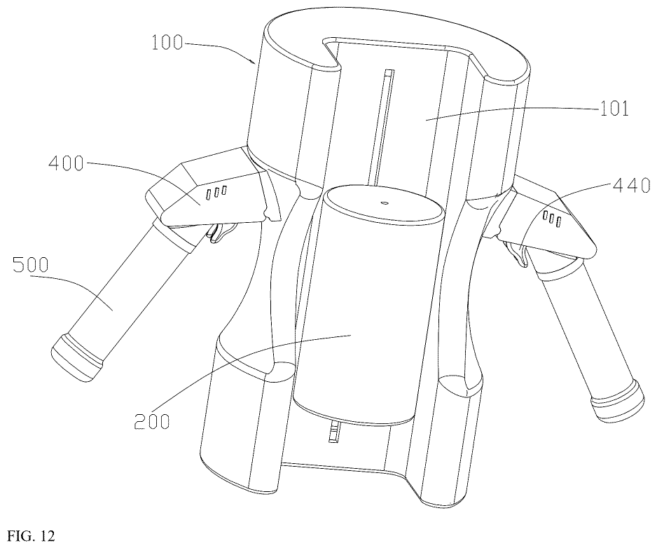

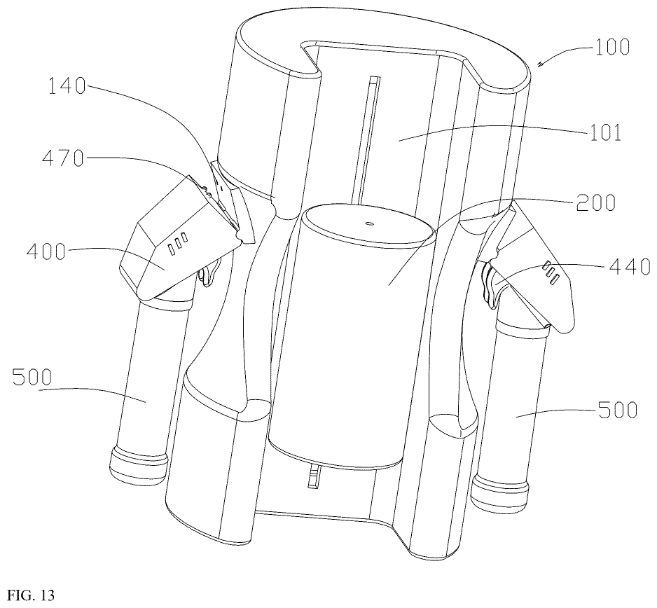

With reference to FIGS. 1 to 13, a sperm collection device, including:
a housing 100, the housing 100 forming a movable space 101;
a sleeve 200, the sleeve 200 being disposed within the movable space 101 and forming a receiving space 201 for accommodating the male genitalia;
a drive assembly 300, the drive assembly 300 being disposed within the housing 100 and driving the sleeve 200 to perform linear reciprocating motion within the movable space 101;
two connection housings 400, the two connection housings 400 being respectively connected to both sides of the outer surface of the housing 100;
two handles 500, the handles 500 being rotatably connected to the connection housings 400 around their axes, and the angle between the axis of the handles 500 and the longitudinal axis of the housing 100 being an acute angle;
a control device 600, the control device 600 being arranged within the connection housings 400 and/or the housing 100, the control device 600 being electrically connected to the drive assembly 300, the control device 600 detecting the rotation angle of at least one of the handles 500 and, based on the rotation angle, controlling the rotation direction and speed of the drive assembly 300 to control the stroke and/or frequency of the reciprocating motion of the sleeve 200.

Through the arrangement of the above structure, when in use, the drive assembly 300 works to drive the sleeve 200 to perform linear reciprocating motion within the movable space 101, thereby massaging and stimulating the penis inserted into the receiving space 201; additionally, the user can rotate the handle 500, the control device 600 detects the rotation angle of the handle 500, and outputs a corresponding electrical signal based on the rotation angle of the handle 500, thereby changing the rotation speed or direction of rotation of the drive assembly 300, ultimately controlling the stroke and/or frequency of the reciprocating motion of the sleeve 200. For example, rotating the left handle 500 can adjust the rotation speed of the drive assembly 300, thereby increasing the frequency of the reciprocating motion of the sleeve 200. Rotating the right handle 500 can adjust the speed of the rotation direction of the drive assembly 300, thereby changing the stroke of the reciprocating motion of the sleeve 200. The user can freely adjust the speed and amplitude of the sleeve 200's motion according to their needs, thus providing a better user experience. Furthermore, since the angle between the axis of the handle 500 and the longitudinal axis of the housing 100 is acute, when the user grips the handle 500, it can prevent the relative rotation between the handle 500 and the housing 100 caused by gravity, allowing the user to more precisely control the rotation angle of the handle 500 and accurately control the stroke and/or frequency of the reciprocating motion of the sleeve 200. Meanwhile, the user feels more comfortable while gripping the product.

In this embodiment, the angle between the axis of the handle 500 and the vertical axis of the housing 100 is between 40° and 80°.

When in use, the user grips the two handles 500 with both hands, with the angle between the axis of the handle 500 and the vertical axis of the housing 100 being between 40° and 80°, which makes the user more comfortable when gripping the handle 500. Furthermore, since there is an angle between the rotation direction of the handle 500 and the movement trend direction generated by the gravity acting on the housing 100, it can prevent the housing 100 from rotating relative to the handle 500 under gravity. This allows the user to more accurately control the rotation angle of the handle 500 relative to the housing 100, making it easier for the user to control the movement mode of the sleeve 200.

In this embodiment, the control device 600 detects the rotation angle of one of the handles 500 and, based on this, controls the rotation direction and speed of the drive assembly 300 to control the stroke of the reciprocating motion of the sleeve 200, while the control device 600 detects the rotation angle of the other handle 500 and, based on this, controls the rotation direction and speed of the drive assembly 300 to control the frequency of the reciprocating motion of the sleeve 200.

In this embodiment, the handle 500 is provided with a magnet 510, and the connection housing 400 is provided with a Hall sensor 410. The Hall sensor 410 is oriented towards the magnet 510 and is used to detect the magnetic field of the magnet 510 and output a voltage signal to the control device 600. Through the structure described above, when the handle 500 rotates relative to the connection housing 400, the position of the magnet 510 relative to the Hall sensor 410 changes, and the Hall sensor 410 detects the change in the magnetic field and generates a corresponding electrical signal. The control device 600 uses this electrical signal to control the speed and direction of rotation of the drive assembly 300. This allows for more accurate detection of the rotation angle of the handle 500 and maps the user's actions to the working mode of the sleeve 200, enhancing the user experience.

In this embodiment, the handle 500 is provided with an axial hole 501, and the connection housing 400 is provided with a rotating shaft 420. The rotating shaft 420 is inserted into the axial hole 501 and is rotatable within the axial hole 501. Through the structure described above, the rotating shaft 420 is inserted into the axial hole 501, enabling the handle 500 to be coaxially arranged with the connection housing 400, and it can rotate relative to it, providing a more stable structure.

In this embodiment, the rotating shaft 420 is provided with an annular protrusion 421, and the inner wall of the axial hole 501 is provided with an annular groove 502. The annular protrusion 421 is inserted into the annular groove 502. By inserting the annular protrusion 421 into the annular groove 502, it prevents relative axial movement between the rotating shaft 420 and the connection housing 400, making the product structure more stable and preventing the rotating shaft 420 from coming out of the axial hole 501.

In this embodiment, the free end of the rotating shaft 420 is provided with a deformation slot 422. When the rotating shaft 420 is inserted into the axial hole 501, by pressing the handle 500 along the axial direction, the inner wall of the axial hole 501 squeezes against the outer wall of the rotating shaft 420 and the outer wall of the annular protrusion 421. The deformation slot 422 allows the rotating shaft 420 to deform, thus enabling the rotating shaft 420 to smoothly insert into the axial hole 501, improving the efficiency of product assembly.

In this embodiment, the connection housing 400 is provided with a limiting block 430, and the handle 500 is provided with an arcuate limiting slot 503. The limiting block 430 is inserted into the limiting slot 503 and can slide within the limiting slot 503. The arcuate limiting slot 503 is arranged on the end face of the handle 500, and the limiting block 430 is arranged on the surface in contact with the end face of the handle 500. The center of the arcuate limiting slot 503 is on the axis of the handle 500. When the user rotates the handle 500, the limiting block 430 slides within the limiting slot 503, and the limiting slot 503 limits the sliding endpoint of the limiting block 430, thus limiting the maximum angle at which the handle 500 can rotate relative to the connection housing 400.

In this embodiment, the control device 600 includes a main circuit board 610 and a button circuit board 620, and the main circuit board 610 is arranged within the housing 100, and the button circuit board 620 is arranged within the connection housing 400, and the main circuit board 610 is electrically connected to the button circuit board 620. The button circuit board 620 is arranged within the connection housing 400 and receives the electrical signal generated by the Hall sensor 410. This electrical signal is then transmitted to the main circuit board 610.

In this embodiment, it further includes a trigger button 440. The trigger button 440 is connected to the connection housing 400 and rotates between a trigger position and a release position. The pressing portion of the trigger button 440 extends out of the connection housing 400. When the pressing portion of the trigger button 440 is driven by external force, the trigger button 440 rotates to the trigger position, and the trigger button 440 presses the key contacts on the button circuit board 620. Through the structure described above, when in use, the user presses the trigger button 440, which rotates from the release position to the trigger position. The trigger button 440 presses the key contacts on the button circuit board 620, generating a corresponding control electrical signal. This enables human-machine interaction, allowing different functions to be realized by pressing different buttons. For example, pressing the trigger button 440 can control the sleeve 200 to vibrate, rotate, or heat, providing users with more functional choices.

In this embodiment, the connection housing 400 is provided with an elastic member 450. One end of the elastic member 450 abuts against the connection housing 400, and the other end of the elastic member 450 abuts against the trigger button 440. The elastic member 450 causes the trigger button 440 to have a tendency to move towards the release position. When the user presses the trigger button 440, it rotates from the release position to the trigger position, causing the elastic member 450 to undergo elastic deformation. When the user releases the trigger button 440, the elastic force of the elastic member 450 drives the trigger button 440 to rotate from the trigger position towards the release position, achieving the reset of the trigger button 440.

In this embodiment, the drive assembly 300 includes a motor 310, a conveyor track 320, a driven gear 330, and a connecting block 340. The motor 310 and the driven gear 330 are disposed within the housing 100, and the conveyor track 320 meshes with the output end of the motor 310 and the driven gear 330. One end of the connecting block 340 is fixedly connected to the conveyor track 320, and the other end of the connecting block 340 extends out of the housing 100 and connects to the sleeve 200. When in use, the motor 310 rotates, driving the conveyor track 320 to move, which in turn drives the connecting block 340 to move. The other end of the connecting block 340 extends out of the housing 100 and connects to the sleeve 200, causing the sleeve 200 to move under the guidance of the connecting block 340. By periodically changing the rotation direction of the motor, the reciprocating motion of the sleeve 200 can be achieved.

In this embodiment, the housing 100 also includes two sliding rods 110, with the two sliding rods 110 positioned on both sides of the connecting block 340. The connecting block 340 is slidably connected to the sliding rods 110. The connecting block 340 is connected to the sliding rods 110 and can slide along them. The sliding rods 110 restrict the movement direction of the connecting block 340, improving the stability of the product and preventing the connecting block 340 from deflecting under the action of driving force.

In this embodiment, the drive assembly 300 further includes a driving gear 350. The motor 310 is fixedly connected to one side of the inner wall of the housing 100, and the output shaft of the motor 310 is rotatably connected to the other side of the inner wall of the housing 100. The driving gear 350 is connected to the middle of the output shaft of the motor 310, and the conveyor track 320 meshes with the driving gear 350. The output shaft of the motor 310 drives the driving gear 350 to rotate, which then meshes with the conveyor track 320, converting the rotation of the driving gear 350 into the linear movement of the conveyor track 320. The conveyor track 320 is track-shaped, and the driving gear 350 and the driven gear 330 are positioned at the two ends of the track-shaped conveyor track 320, ensuring the stable transmission of power.

In this embodiment, the housing 100 also includes an output shaft fixing member 120.

The output shaft fixing member 120 is fixedly connected to the inner wall of the housing 100 and connects to the output shaft of the motor 310 to prevent radial movement of the motor's output shaft. The output shaft fixing member 120 is connected to the inner wall of the housing 100, fixing the output shaft of the motor 310, further enhancing the stability of the product and providing support for the motor's output shaft in various axial positions, preventing deformation of the output shaft.

In this embodiment, the outer surface of the housing 100 is provided with a connecting groove 102. The connection housing 400 can be detachably inserted into the connecting groove 102. With the structure described above, when storing the product, the connection housing 400 and the handle 500 can be removed to reduce the transportation and storage volume. When in use, the connection housing 400 is inserted into the connecting groove 102 to assemble the product.

In this embodiment, the connection housing 400 is provided with a first electrical connection terminal 460, and the connecting groove 102 is provided with a second electrical connection terminal 130. The first electrical connection terminal 460 is electrically connected to the second electrical connection terminal 130. With the structure described above, when the connection housing 400 is inserted into the connecting groove 102, the first electrical connection terminal 460 is electrically connected to the second electrical connection terminal 130, achieving stable electrical connection between the main circuit board 610 and the button circuit board 620.

In this embodiment, the connection housing 400 is connected to the housing 100 and rotates between a use position and a storage position. With the structure described above, in this embodiment, the connection housing 400 is rotatably connected to the housing 100. When in use, the connection housing 400 can be rotated to the use position for easy handling by the user, and when storing the product, it can be rotated to the storage position, bringing the connection housing 400 close to the housing 100, reducing the width of the product for easy transportation.

In this embodiment, the connection housing 400 is provided with a third electrical connection terminal 470, and the connecting groove 102 is provided with a fourth electrical connection terminal 140. The third electrical connection terminal 470 is electrically connected to the fourth electrical connection terminal 140. With the structure described above, when the connection housing 400 is rotated to the use position, the third electrical connection terminal 470 is electrically connected to the fourth electrical connection terminal 140, achieving stable electrical connection between the main circuit board 610 and the button circuit board 620.

1. A sperm collection device, comprising:
a housing, the housing forming a movable space;
a sleeve, the sleeve being disposed within the movable space and forming a receiving space for accommodating the male genitalia;
a drive assembly, the drive assembly being disposed within the housing and driving the sleeve to perform linear reciprocating motion within the movable space;
two connection housings, the two connection housings being respectively connected to both sides of an outer surface of the housing;
two handles, the two handles being rotatably connected to the two connection housings around their axes, and the angle between an axis of each of the two handles and the vertical axis of the housing is from 40° to 80°;
a control device, the control device being arranged within at least one of the two connection: housings and/er the housing, the control device being electrically connected to the drive assembly, the control device detecting a rotation angle of at least one of the two handles and controlling the rotation direction and speed of the drive assembly based on the rotation angle, to control at least one of the stroke and frequency of the reciprocating motion of the sleeve:
wherein each of the two handles is provided with a magnet. each of the two connection housings is provided with a Hall sensor, the Hall sensor being oriented towards the magnet, the. Hall sensor being used to detect the magnetic field of the magnet and output a voltage signal to the control device.

2. (canceled)

3. The sperm collection device according to claim 1, wherein the control device detects the rotation angle of one of the two handles and controls the rotation direction and speed of the drive assembly based on the rotation angle to control the stroke of the reciprocating motion of the sleeve, the control device detecting the rotation angle of the other handle and controlling the rotation direction and speed of the drive assembly based on the rotation angle to control the frequency of the reciprocating motion of the sleeve.

4. (canceled)

5. The sperm collection device according to claim 1, wherein each of the two handles is provided with an axial hole, each of the two connection housings is provided with a rotating shaft, the rotating shaft being inserted into the axial hole and rotatable within the axial hole.

6. The sperm collection device according to claim 5, wherein the rotating shaft is provided with an annular protrusion, an inner wall of the axial hole being provided with an annular groove, the annular protrusion being inserted into the annular groove.

7. The sperm collection device according to claim 5, wherein a free end of the rotating shaft is provided with a deformation slot.

8. The sperm collection device according to claim 5, wherein each of the two connection housings is provided with a limiting block, each of the two handles is provided with an arcuate limiting slot, the limiting block being inserted into the limiting slot and being slidable within the limiting slot.

9. The sperm collection device according to claim 1, wherein the control device comprises a main circuit board and a button circuit board, the main circuit board being arranged within the housing, the button circuit board being arranged within one of the two connection housings, the main circuit board being electrically connected to the button circuit board.

10. The sperm collection device according to claim 9, further comprising a trigger button, the trigger button being connected to the one of the two connection housings and rotating between a trigger position and a release position, the pressing portion of the trigger button extending out of the correspondingly connected one of the two connection housings, and when the pressing portion of the trigger button is pressed by an external force, the trigger button rotates to the trigger position, pressing the key contacts on the button circuit board.

11. The sperm collection device according to claim 10, wherein an elastic member is disposed in each of the two connection housings, one end of the elastic member abutting against the corresponding one of the two connection housings and the other end of the elastic member abutting against the trigger button, the elastic member biasing the trigger button toward the release position,

12. The sperm collection device according to claim 1, wherein the drive assembly comprises a motor, a conveyor track, a driven gear, and a connecting block, the motor and the driven gear being disposed within the housing, the conveyor track meshing with an output end of the motor and the driven gear, one end of the connecting block being fixedly connected to the conveyor track, and the other end of the connecting block extending out of the housing and being connected to the sleeve.

13. The sperm collection device according to claim 12, wherein the housing further comprises two sliding rods, the two sliding rods being respectively located on both sides of the connecting block, and the connecting block being slidably connected to the sliding rods.

14. The sperm collection device according to claim 12, wherein the drive assembly further comprises a driving gear, the motor being fixedly connected to one side of an inner wall of the housing, an output shaft of the motor being rotatably connected to the other side of the inner wall of the housing, the driving gear being connected to a middle portion of the output shaft of the motor, and the conveyor track meshing with the driving gear.

15. The sperm collection device according to claim 13, wherein the housing further comprises an output shaft fixing member, the output shaft fixing member being fixedly connected to an inner wall of the housing and connected to the output shaft of the motor so as to restrain radial movement of the output shaft of the motor.

16. The sperm collection device according to claim 1, wherein an outer surface of the housing is provided with two connecting grooves, and the two connection housings are detachably inserted in the two connecting grooves.

17. The sperm collection device according to claim 16, wherein a first electrical connection terminal is provided on a side of each of the two connection housings facing the housing, a second electrical connection terminal is provided in each of the two connecting grooves, and the first electrical connection terminal is electrically connected to the second electrical connection terminal.

18. The sperm collection device according to claim 1, wherein each of the two connection housings is connected to the housing and rotates between a use position and a storage position,

19. The sperm collection device according to claim 18, wherein a third electrical connection terminal is provided on a side of each of the two connection housings facing the housing, a fourth electrical connection terminal is provided in each of the two connecting grooves, and the third electrical connection terminal is electrically connected to the fourth electrical connection terminal.
